(19) United States
(12) Patent Application Publication
(10) Pub. No.: US 2019/0038437 A1
(43) Pub. Date: Feb. 7, 2019

(54) LAMINATED SPRING CONTAINING VALVE, CONTROL METHOD OF THIS VALVE AND PROSTHETIC JOINT THAT INCLUDES THE VALVE

(71) Applicants: BAMA TEKNOLOJI TIBBI CIHAZLAR DANISMANLIK SAGLIK B ILISIM SANAYI VE TICARET LIMITED SIRKETI, Ankara (TR); ORTOTEK ORTOPEDI PROTEZ ORTEZ REHABILITASYON MERKE ZI TICARET LIMITED SIRKETI, Ankara (TR)

(72) Inventors: Akay OZTURK, Ankara (TR); Can Mehmet Ali CIFTCI, Ankara (TR)

(73) Assignees: BAMA TEKNOLOJI TIBBI CIHAZLAR DANISMANLIK SAGLIK BILISIM SANAYI VE TICARET LIMITED SIRKETI, Ankara (TR); ORTOTEK ORTOPEDI PROTEZ ORTEZ REHABILITASYON MERKEZI TICARET LIMITED SIRKETI, Ankara (TR)

(21) Appl. No.: 15/764,883

(22) PCT Filed: Sep. 30, 2016
(86) PCT No.: PCT/TR2016/050361

§ 371 Date: Mar. 30, 2018

(30) Foreign Application Priority Data
Sep. 30, 2015 (TR) 2015/12082
Sep. 30, 2015 (TR) 2015/12083
Dec. 17, 2015 (TR) 2015/16301
Dec. 17, 2015 (TR) 2015/16302
Dec. 17, 2015 (TR) 2015/16303

(51) Int. Cl. A61F 2/68; F16F 9/348; F16F 9/46

(52) U.S. Cl. CPC A61F 2/68; A61F 2002/741; F16F 9/467; F16F 9/3488

(57) ABSTRACT

Present invention concerns a laminated spring containing valve on a cylinder that contains two compartments which are separated by a piston, which ensures the adjustment of the fluid flow rate between the two compartments by being sensitive to the pressure difference between the two compartment. Present invention explains a valve and its control method which provides a dynamical control over the fluid flow between the two compartments and the flow rate. Besides, this invention explains a valve set and a prosthetic joint, which contains this set, that ensures the adaptive movement of the prosthetic limbs of the amputees with the natural movement phases.

### CROSS REFERENCE TO RELATED APPLICATIONS

This application is the national phase entry of International Application No. PCT/TR2016/050361, filed on Sep. 30, 2016, which is based upon and claims priority to Turkish Application No. 2015/12082 filed on Sep. 30, 2015, Turkish Application No. 2015/12083 filed on Sep. 30, 2015, Turkish Application No. 2015/16301 filed on Dec. 17, 2015, Turkish Application No. 2015/16302 filed on Dec. 17, 2015, Turkish Application No. 2015/16303 filed on Dec. 17, 2015, the entire contents of which are incorporated herein by reference.

### TECHNICAL FIELD OF THE INVENTION

Present invention is related to a cylinder that includes two compartments separated by a piston, a laminated spring valve that allows the regulation of fluid flow rate between the compartments in a way sensitive to the pressure difference between closets, control of the fluid flow rate in a dynamic way by this valve and a prosthetic joint that includes the valve set in which the fluid flow and fluid flow rate is controlled in a dynamic way.

### BACKGROUND OF THE INVENTION

In piston systems, the pressures of the compartments defined by the piston are changed as a result of the piston during the operation of the system and this change forms resistance against the movement of the piston. In various applications, the alteration of the compartment pressures must be performed for the realization of the piston movement in a desired way. For example, after the pressure in a compartment that is compressed by the piston exceeded a certain value, reduction of the pressure that is faced by the piston during its movement or protection of the system from high pressure must be ensured by allowing the fluid flow from this compartment to other compartment. Thus, a piston that moves with different velocities or different amounts in the situation when the exerted force remained under or above a certain threshold value can be obtained.

Various valves are known for attainment of pistons according to these descriptions.

A moving valve element between two laminated springs that contains at least one opening that allows the liquid flow was explained in document numbered JPH05164174A. This valve element presses one of the laminated springs depending on piston movement and as a consequence liquid passage in that direction will be provided

A shock absorber that includes openings that allow the outward fluid flow for the sake of protection when faced with a pressure more than expected was explained in document numbered FR2325856A1. The openings are covered with spring made from sheet for closure of the openings during normal operation and opening of them under high pressure.

A shock absorber that the opening on the piston is closed by a plug that is located at the tip of the laminated spring that is located at the compartment that is compressed by the piston as a consequence of vibration and opening of the hole with the cessation of the compressing movement and pressure between two compartments are equalized in the document numbered CN201568515U.

Amputation is process that is defined as a complete or partial removal of a limb of person from his/her body as a result of medical reasons or accidents. This operation can be applied with therapeutic reasons as a consequence of the disease of a person and also occur as a consequence of an accident after which the replantation process cannot be performed. People whom replantation cannot be performed on, in the case of limb loss are called amputees. Prostheses that are obtained from different materials and different mechanisms are used for movement of these people without requiring help during the history. First prostheses in history generally produced from inelastic wooden materials and developed for small limbs such as toes. Before that it is thought that sticks were used in place of lost limbs. However, different prostheses for different limbs are developed in our day for the comfortable living of amputees.

Especially development of up knee prostheses that allow the walking of amputees whose legs up to knee is amputated without using any stick or any other support, constitutes one of the important research and development areas in our day. Many different prosthetic knee joints oriented to various needs and requests of the users are present in the Prior Art. Knee prosthesis forms a resistance that allows the completion of near-normal movement, during the forward movement of the lower leg and remaining behind while pressing to the ground like the normal situation in order to mimic the normal movement of the lower leg and knee joint.

Knee joints that are used in the up-knee prostheses separated into five according to their ability to form movement resistance. These are mechanical systems, hydraulic systems, pneumatic systems, hybrid systems which pneumatic and hydraulic systems are used together and magneto-rhelogic systems which the magneto-rhelogic liquids are used. Besides, systems also divided into two according to adjustment of resistances of these systems as passive and active systems. In mechanical systems, the movement of up-knee prosthesis can be controlled with mechanical frictions of joint components.

In hydraulic systems, the inflexion and flexion resistances during the movement of artificial knee joint are adjusted by alteration of cross-sectional areas of channel between two different components. Force is transmitted with hydraulic principles during the movement of the joint. In pneumatic systems, force is transmitted with the gases. In hybrid systems, the mutual usages of these two force transfer mediums are present. Passive controlled and microprocessor controlled applications of these artificial knee joints which are separated on their force transfer mediums are present.

Hydraulic, pneumatic and hybrid joint a system that are known in the art, occupies too much space especially to form adequate resistance against the flexion of the knee, and thus cannot be placed upon the prosthesis ergonomically. Besides, because these non-compact systems have a center of gravity which is non-suitable for the original knee, prevents the healthy walking of the user.

A compact prosthetic knee joint that is developed to address these problems is seen in the patent documents numbered TR201410592 and WO2016036336.

### SUMMARY OF THE INVENTION

Aim of this invention is development of a valve that contains a laminated spring, which allows the adjustment of fluid flow rate according to the pressure difference between two compartments in cylinder which includes two compartments separated with a piston. With this invention, development of a laminated spring containing valve that allows the fluid flow with a predetermined velocity in one side and the fluid flow with different velocities under and over a specific pressure difference on the other side. Selection of the aforesaid pressure difference as different for both of the direction is also possible for this invention.

Another aim of this invention is development of a valve that allows the control of the passage velocity and fluid flow between two compartments dynamically in cylinder containing two compartments that are separated by a piston and control method of this valve.

Yet another aim of this invention is development of a valve set that can direct the fluids that form resistance during the movement of prosthesis parts that corresponds the bones, as compatible with the natural movement stages of the prosthesis and a prosthetic joint that includes valve set.

In line with the aims of the invention, a valve that includes a laminated spring that covers the opening of the hole and a hole which is located on the piston that connects the two compartments are developed. This hole is overlying on a curve around the center on the piston, continuously or non-continuously. Laminated spring is attached to the surface of the piston from the aforesaid center. Spring does not contains any part that has a shape and area which can cover the hole and located in a distance to hole that it can cover the hole.

According to the present invention, valve is operated as both an adjustment valve and also a check valve.

With present invention, also a piston which contains at least one valve according to the invention is developed. Besides, a piston which contains at least two opposite oriented valve according to the invention is also developed.

Again, for the purposes of present invention a hole which connects two compartments to each other and located upon the surface of the piston and a valve that includes two laminated springs that covers the openings of this hole facing the both compartments are developed. Laminated springs are attached to the surface of the piston. Springs contains a portion that at least covers the hole completely throughout a circle whose center is the attachment point which they attached and passes the hole and another portion which is adjacent to this area and whose radius is narrowed in the direction of azimuth in a way it never covers the hole.

Yet again, for the purposes of present invention, a valve that includes a channel that allows the passage of fluids between two compartments, a laminated spring that can cover the opening of this channel partially or completely and an motor that controls the fluid flow and/or fluid flow rate by directing the aforesaid spring to different angular positions, is developed. Spring contains a portion that covers the opening of the channel at least and an opening and the complete coverage or partial coverage of the channel opening by the spring can be realized by alteration of the angular position of the spring by the motor.

Again, for the purposes of present invention, a valve set that contain two channels that allow the fluid flow between two compartments, two laminated spring that can cover the openings of these channels completely or partially and an motor that control the fluid flow and/or fluid flow rate by directing these spring to different angular locations, is developed. Springs contains a portion which can at least cover the related channels opening completely and an gap and the complete coverage or partial coverage of the channel opening by the spring can be realized by alteration of the angular position of the spring by the motor.

With present invention, also a control method, in which a valve set according to the invention will be worked in a way that will confront pressure that is changed according to the movement phase of the piston, is developed.

Yet again, the valve set developed for the purposes of present invention, is used in prosthetic knee joints that contain two fluid compartments separated by a piston from each other in a cylinder and turn the angular movement of the parts of the prosthesis against each other to linear movement of cylinder and piston against each other in a way that change the dimensions of the fluid compartments. Valve set allows the adjustment of the pressure that is affected the piston because of the fluid pressure, by adjusting the fluid flow and fluid flow rate between the compartments. Thus, the movement of the prosthetic parts against each other can be limited in a desired way that is suitable for the natural movement of limb that the prosthesis is used in place for.

In one embodiment of the present invention, prosthetic joint of invention contains a valve set that adjust the pressure over a piston that is attached to the prosthetic parts from a point which is not lying on the rotation axis of the prosthetic joint during the angular movement of the prosthetic parts against each other or a piston that is pressed by a fluid during the angular movement of prosthetic parts against each other.

In one preferred embodiment of the present invention, prosthetic joint of invention contains a cylinder that moves in a linear route along the rotation axis according to the piston by interacting with the teeth's that lies perpendicular to the rotation axis and placed upon one of the prosthetic parts, during the angular movement of the prosthetic parts against relative to each other and a piston located on the rotation axis that passes over the attachment point of the prosthetic parts and attached to the one of the prosthetic parts.

### DETAILED DESCRIPTION OF THE DRAWINGS

Figures and related explanations used for better explanation of the laminated spring containing valve, its control method and prosthetic joint contains this valve developed with the present invention is shown below.

FIG. 53. Isometric partial section appearance of system consists of piston and cylinder according to an embodiment of present invention.

FIG. 54. Partial section appearance of the piston system consists of piston and cylinder from side.

FIG. 55. Partial section appearance of the piston from side.

FIG. 56. Isometric partial section appearance of piston system consists of piston and cylinder according to an embodiment of present invention.

FIG. 57. Partial section appearance of the piston system consists of piston and cylinder from side.

FIG. 58. Partial section appearance of the piston from side.

FIG. 59. Plan view of the valve in an embodiment which the strings are placed in a two different angular position.

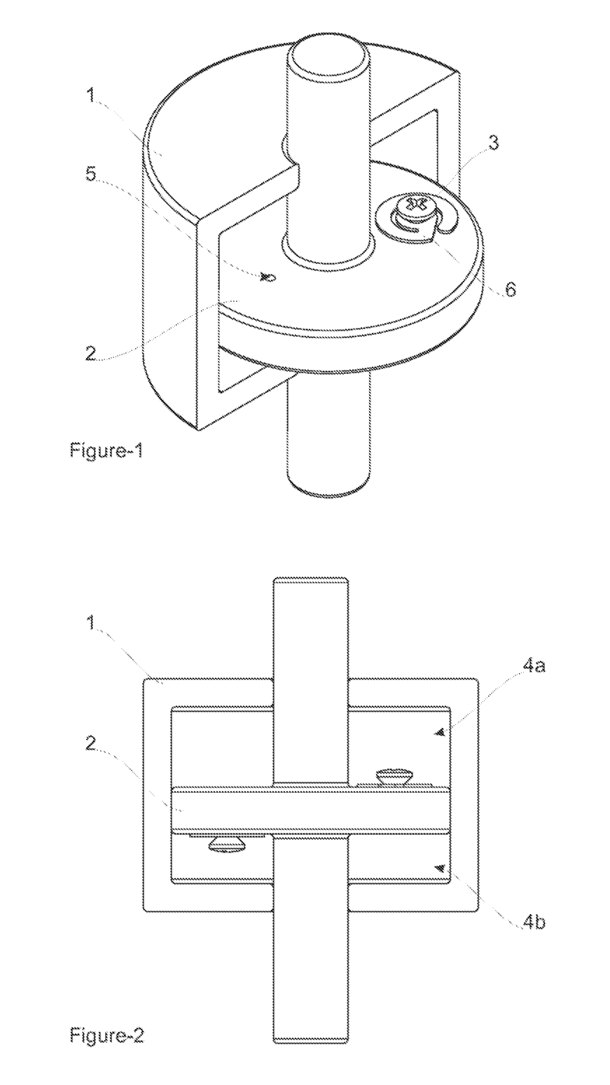

FIG. 60. Plan view of the valve at FIG. 7 in which also the second spring is shown with discrete representation.

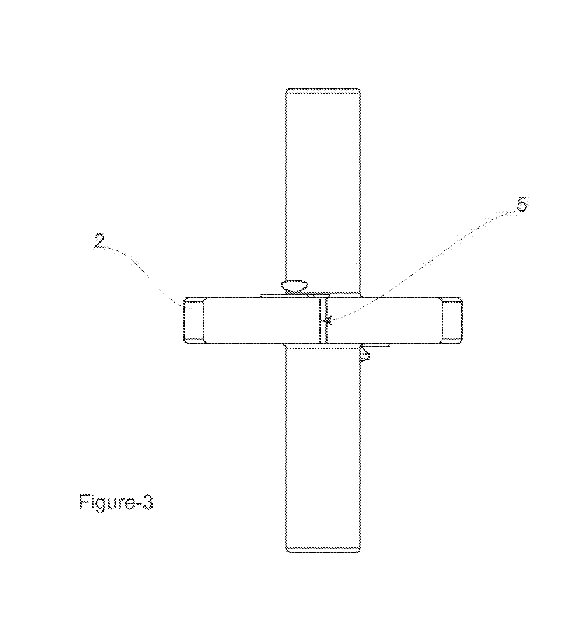

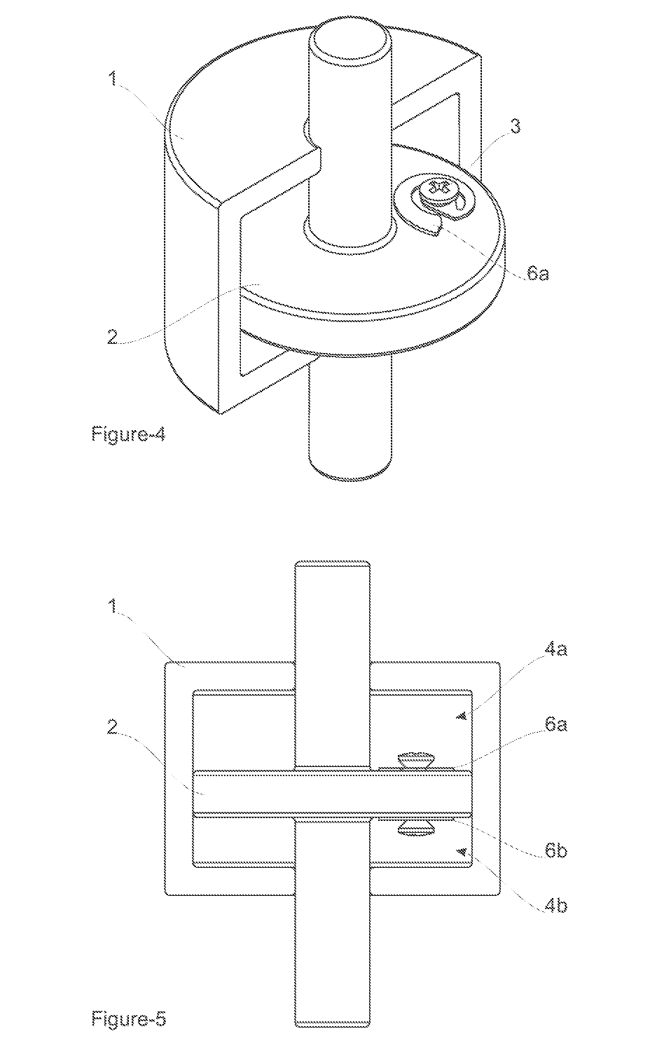

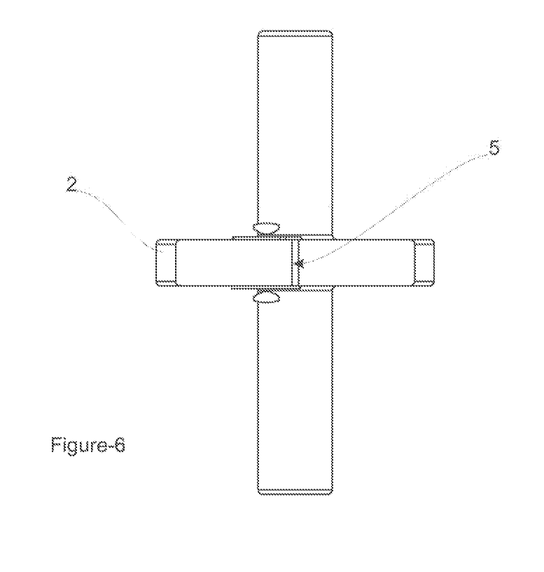

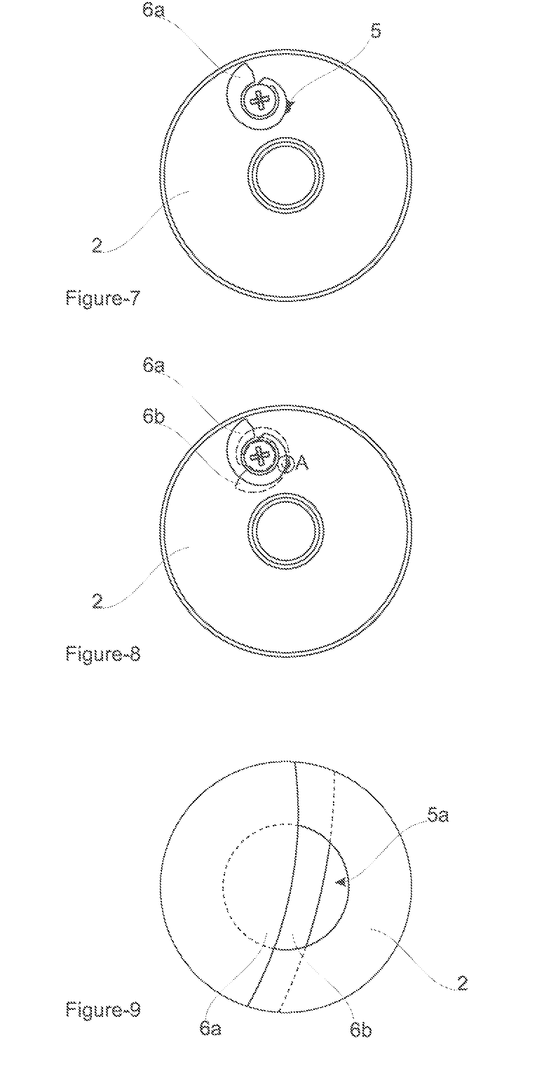

FIG. 61. Appearance of the A detail of FIG. 8.

FIG. 62. Plan view of a continuous hole that can be used with the valve of the present invention.

FIG. 63. Plan view of a non-continuous hole that can be used with the valve of the present invention.

FIG. 64. Plan view of a hole that can be used with the valve of the present invention.

FIG. 65. Plan view of a hole that can be used with the valve of the present invention.

FIG. 66. Plan view of a spring that can be used with the valve of the present invention.

FIG. 67. Plan view of a spring that can be used with the valve of the present invention.

FIG. 68. Plan view of a spring that can be used with the valve of the present invention.

FIG. 69. Isometric appearance of the piston according to an embodiment of present invention.

FIG. 70. Plan view of the piston.

FIG. 71. Base view of the piston.

FIG. 72. Plan view of the piston without the motor and cover.

FIG. 73. Plan view of the piston without the motor, cover, transmission shaft and spring.

FIG. 74. Side view of the piston.

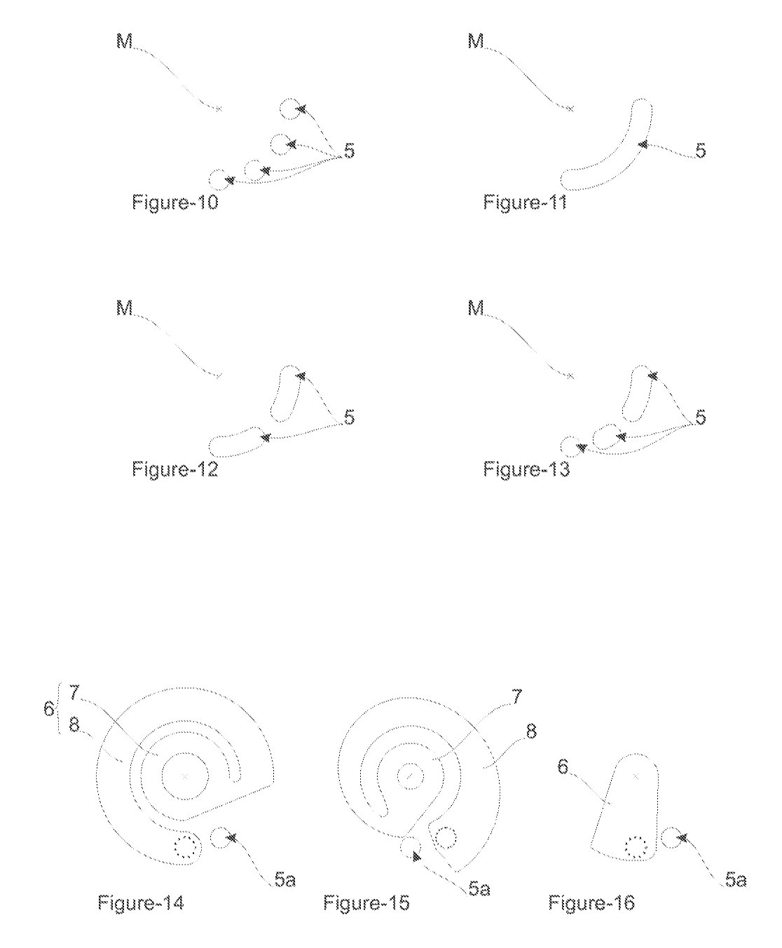

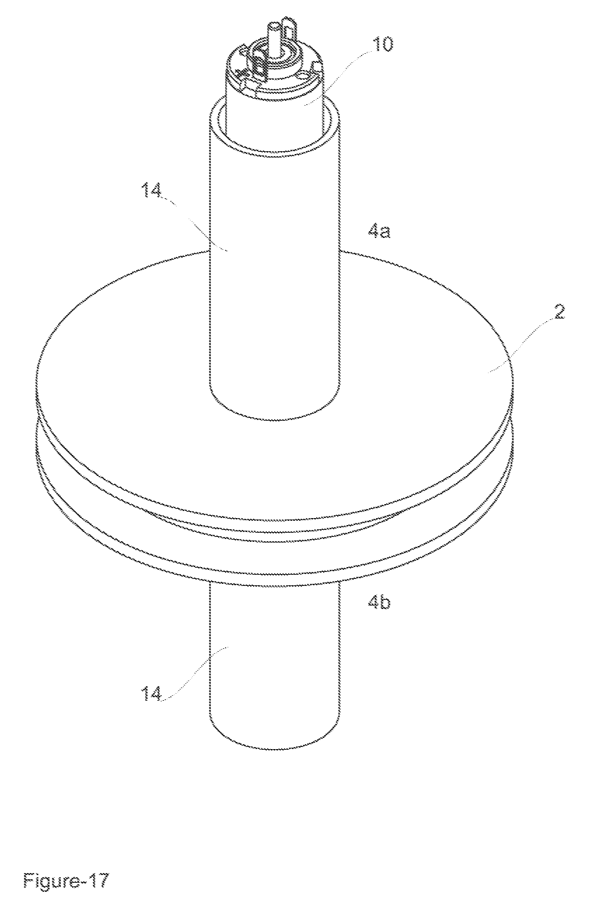

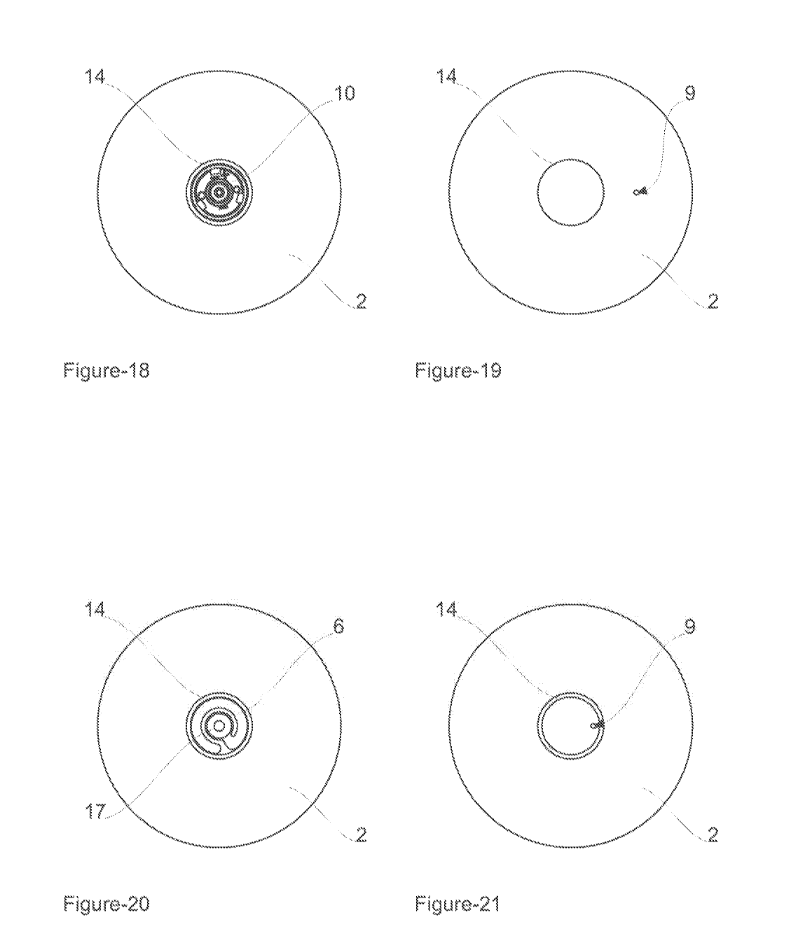

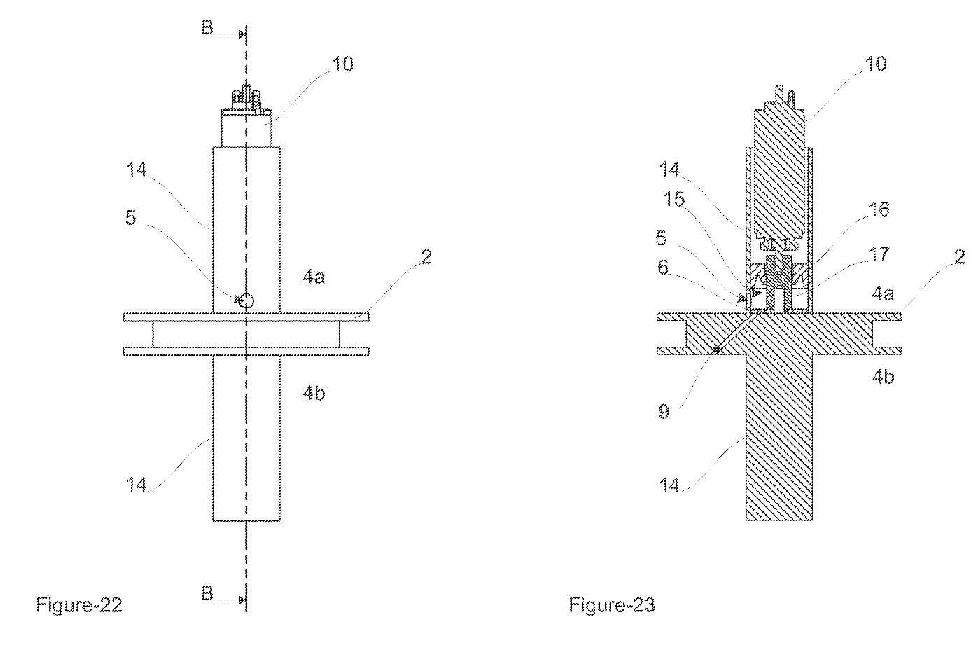

FIG. 75. Side view of the piston according to the B-B section line at FIG. 22.

FIG. 76. Isometric appearance of the piston according to an embodiment of present invention.

FIG. 77. Plan view of the piston.

FIG. 78. Base view of the piston.

FIG. 79. Plan view of the piston without motor and gasket.

FIG. 80. Plan view of the piston without motor, gasket, transmission shaft and spring.

FIG. 81. Side view of the piston.

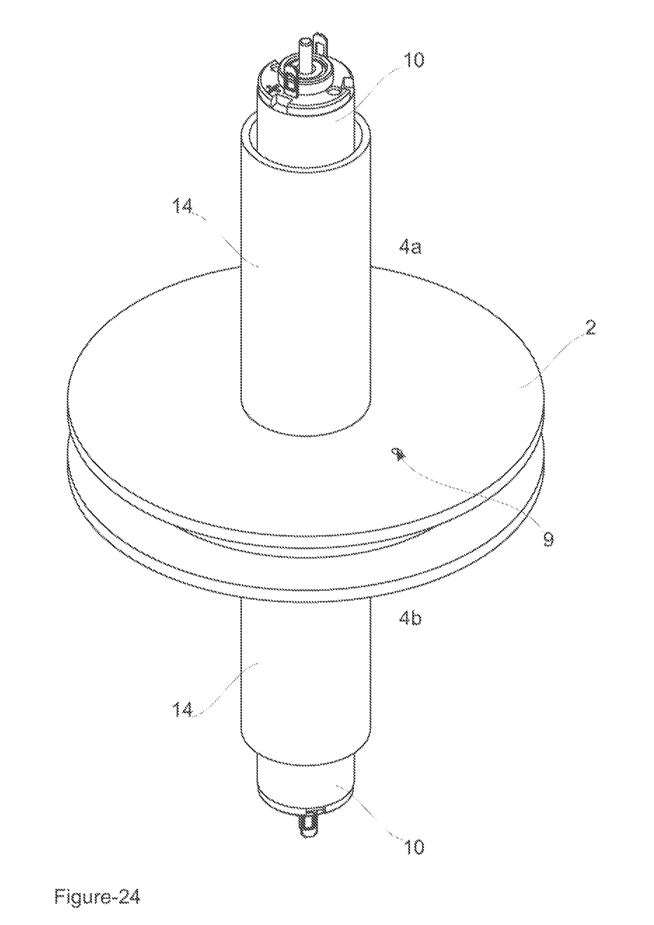

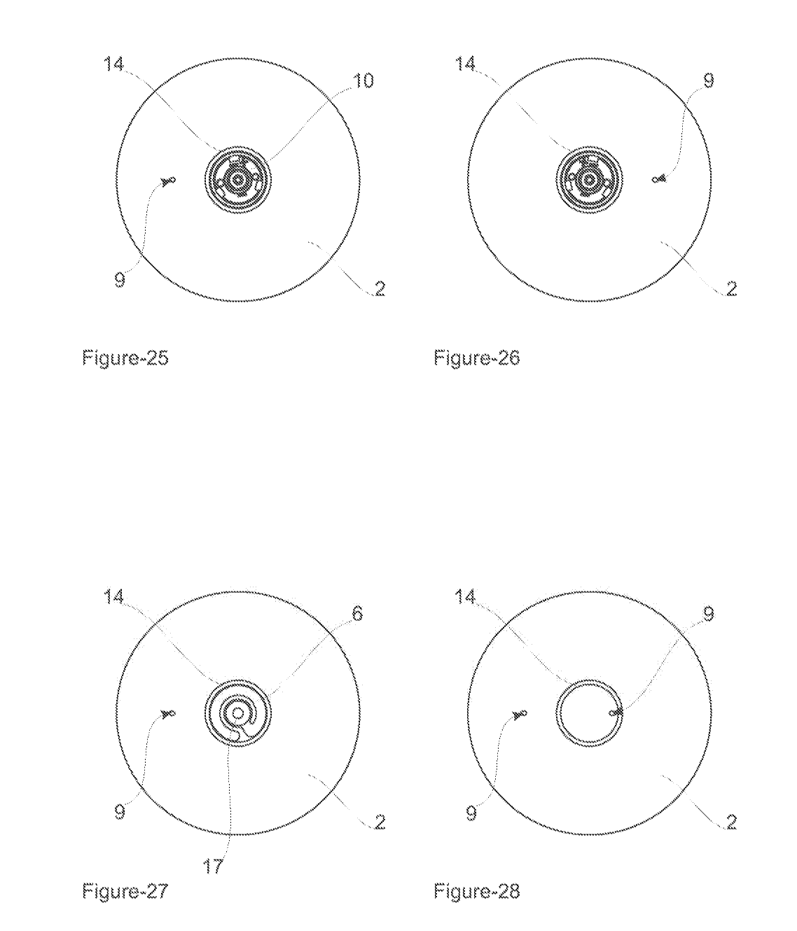

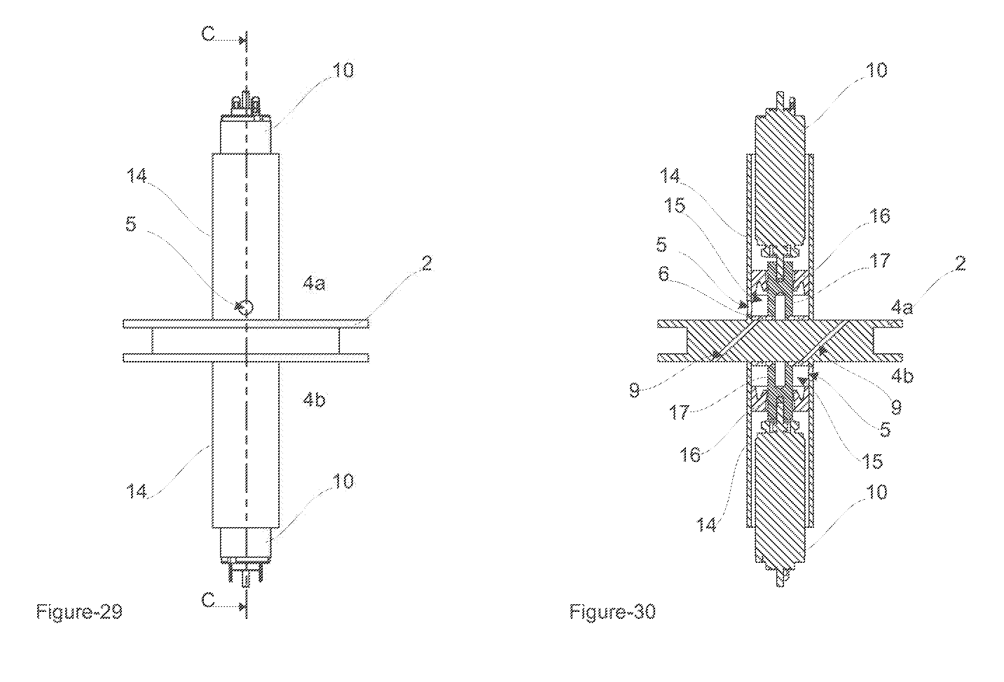

FIG. 82. Side sectional view of the piston according to C-C section line of FIG. 29.

FIG. 83. Side sectional view of the piston according to an embodiment of the present invention.

FIG. 84. Flow diagram of the control method of present invention.

FIG. 85. Plan view of a spring that can be used with a valve set of present invention in an angular position that covers the opening of the channel completely.

FIG. 86. Plan view of a spring that can be used with a valve set of present invention in an angular position that covers the opening of the channel partially.

FIG. 87. Plan view of a spring that can be used with a valve set of present invention in an angular position that doesn't covers the opening of the channel at all.

FIG. 88. Plan view of another spring that can be used with a valve set of present invention.

FIG. 89. Prosthetic appearance of a prosthetic joint of present invention.

FIG. 90. Isometric appearance of the cylinder.

FIG. 91. Isometric appearance of the cylinder.

FIG. 92. Plan view of the cylinder.

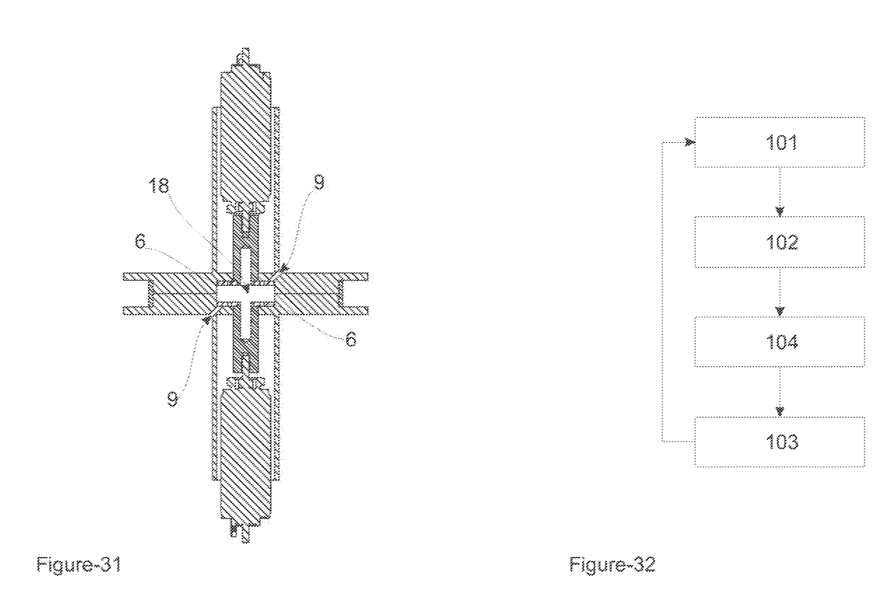

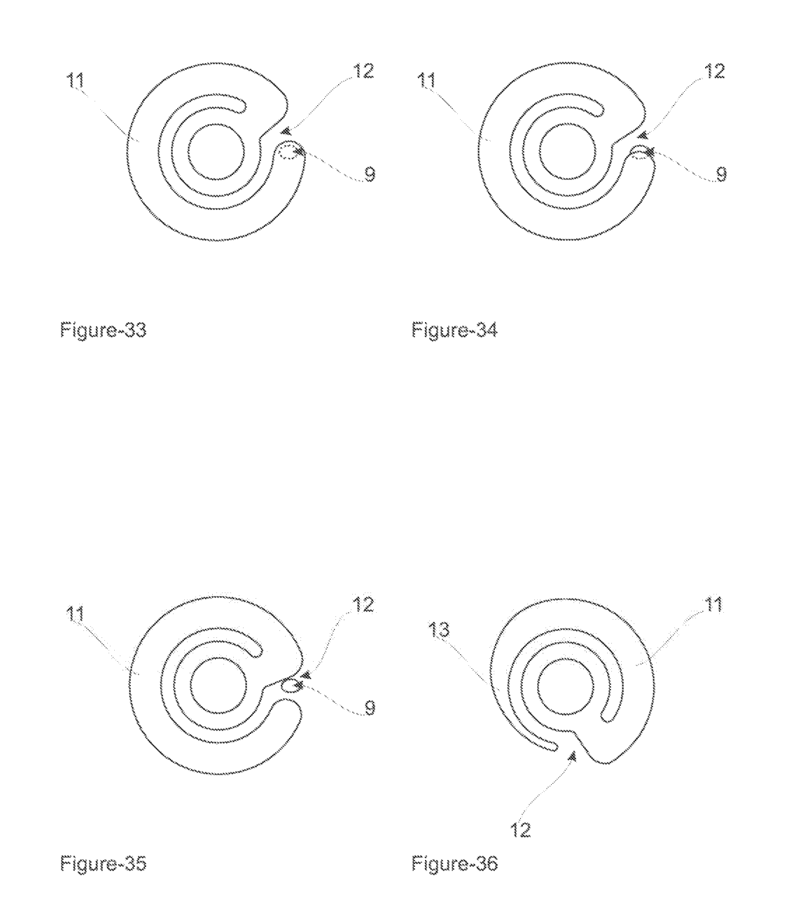

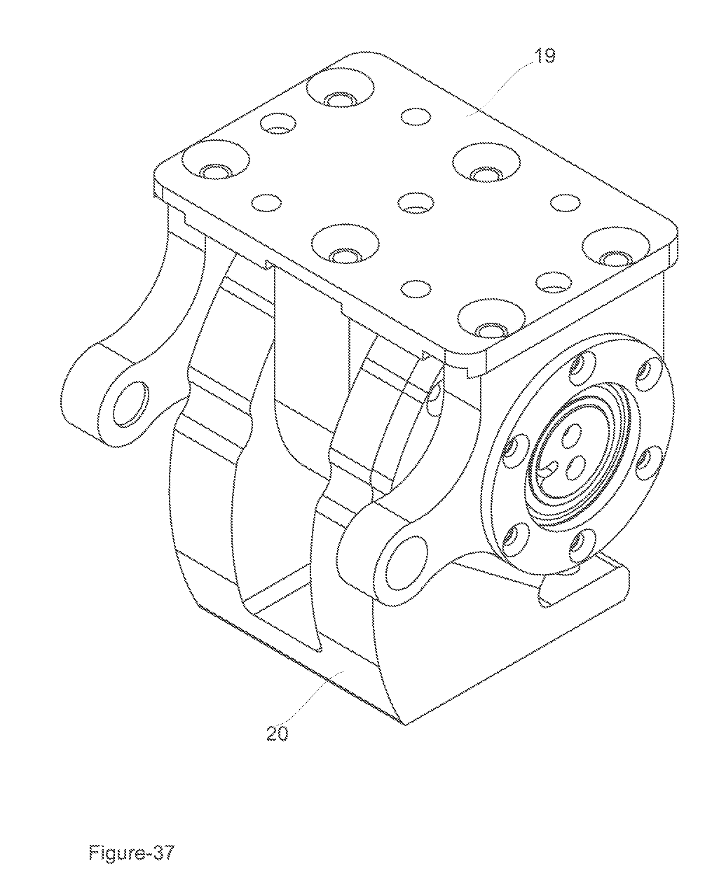

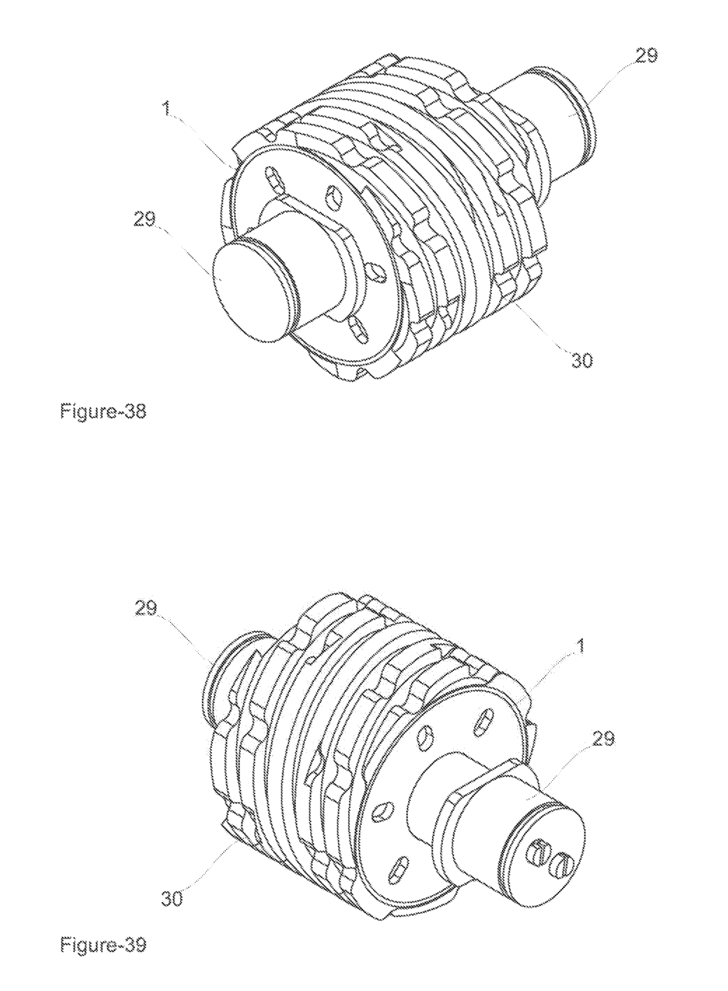

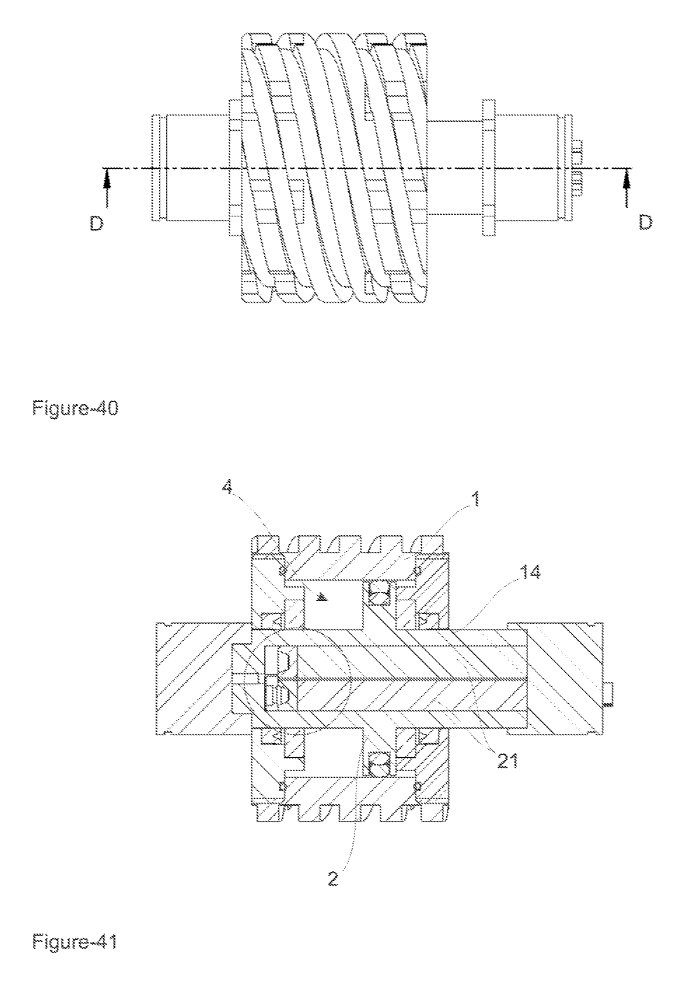

FIG. 93. Sectional view of the cylinder according to the D-D sectional line of FIG. 40.

FIG. 94. Detailed appearance of marked area at FIG. 41.

FIG. 95. Isometric appearance of the extension of the second body that bears the cylinder.

FIG. 96. Isometric appearance of the extensions of the first body that bears the cylinder.

FIG. 97. Isometric appearance of piston and shaft.

FIG. 98. Isometric appearance of piston and shaft.

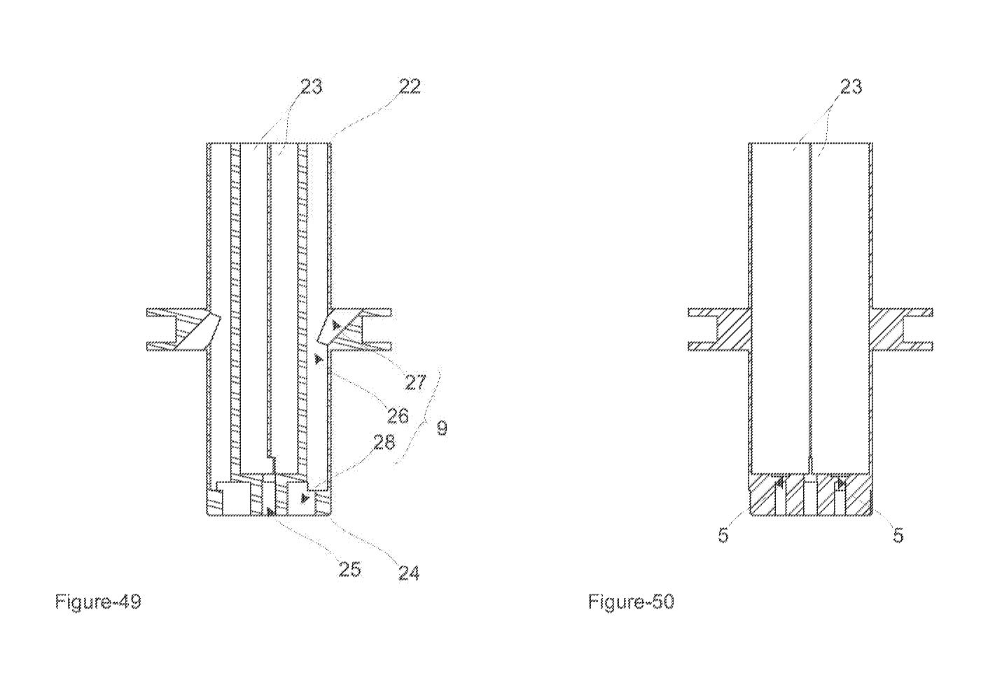

FIG. 99. Appearance of piston and shaft from the side first end.

FIG. 100. Appearance of piston and shaft from the side second end.

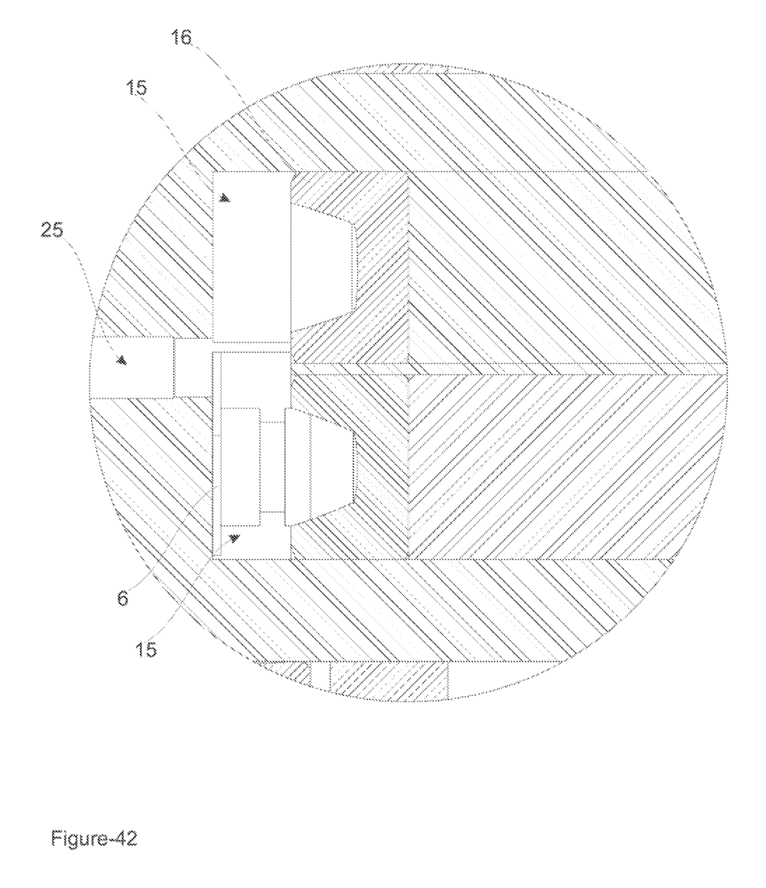

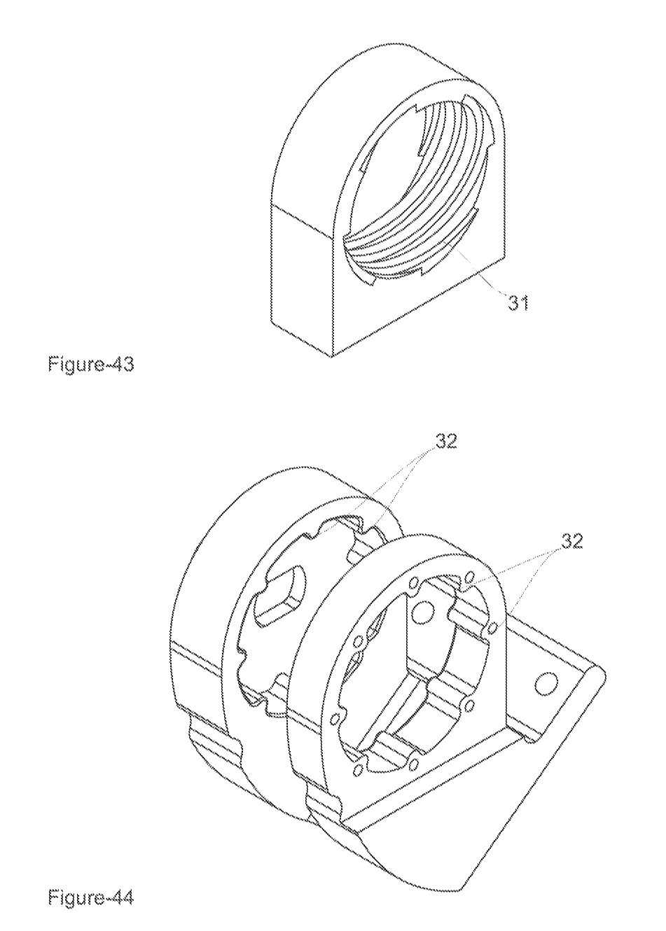

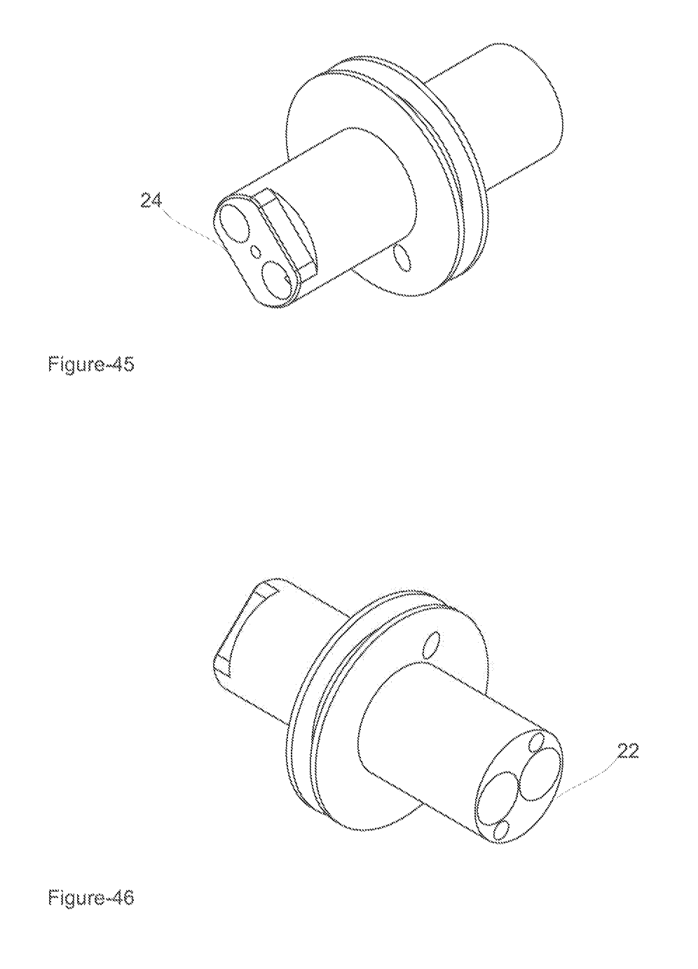

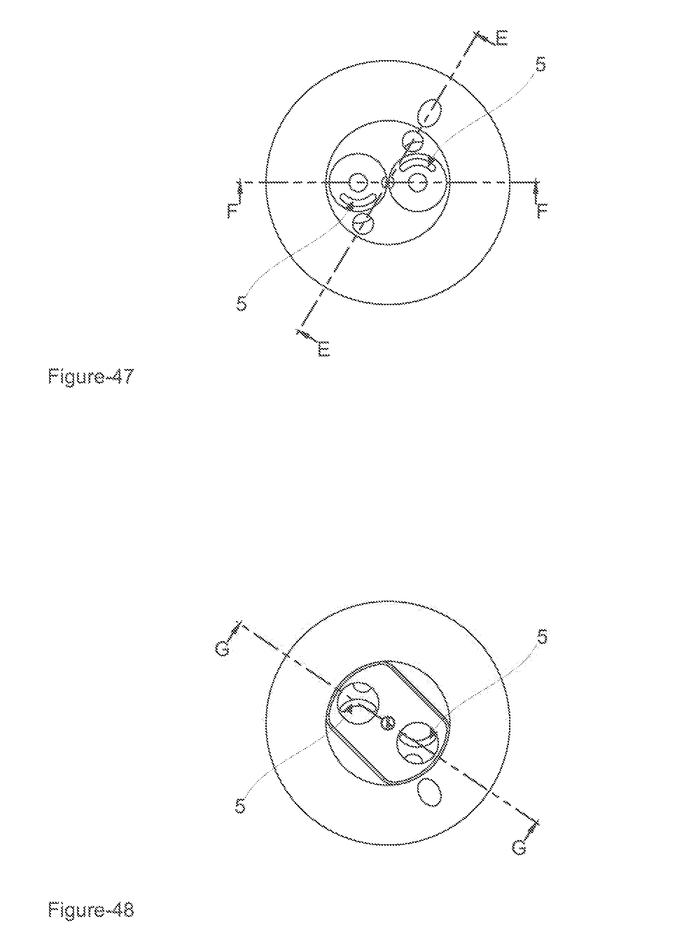

FIG. 101. Sectional view of the piston and shaft according to E-E sectional line of FIG. 47.

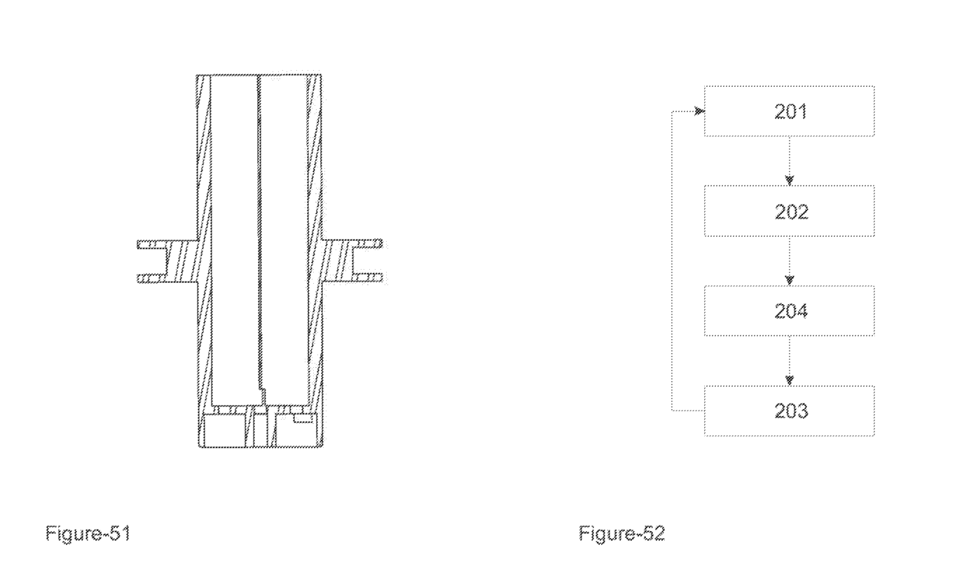

FIG. 102. Sectional view of the piston and shaft according to F-F sectional line of FIG. 47.

FIG. 103. Sectional view of the piston and shaft according to G-G sectional line of FIG. 48.

FIG. 104. Flow diagram about the control of prosthetic joint.

### DETAILED DESCRIPTIONS OF THE ELEMENTS THAT FORM THE PRESENT INVENTION

For better explanation of the laminated spring containing valve developed with present invention, control method of this valve and the prosthetic joint containing this valve, the parts and portions are numbered and corresponding explanations are provided below.
1. Cylinder
2. Piston
3. Screw
4. Compartment
4a. First compartment
4b. Second compartment
5. Hole
5a. Effective aperture
6. Spring
6a. First spring
6b. Second spring
7. Central sheet
8. Spiral arm
9. Channel
10. Motor
11. Covering portion
12. Gap
13. Partially covering portion
14. Shaft
15. Intermediate compartment
16. Gasket
17. Transmission shaft
18. Common intermediate compartment
19. First body
20. Second body
21. Actuator
22. First end
23. Actuator bed
24. Second end
25. Groove
26. First part
27. Second part
28. Third part
29. Cover
30. Teeth
31. Counter Teeth
32. Linear Counter Teeth
M. Center

### DETAILED DESCRIPTION OF THE INVENTION

Valve of present invention, provides the fluid flow between a first compartment (4a) and second compartment (4b) separated by piston (2) which is placed into a cylinder (1) and that can move both directions along the axis of the cylinder (1) during the movement of the piston (2). Valve, contains at least one hole (5) that is placed on the piston (2) and allows the fluid flow between first compartment (4a) and second compartment (4b) and a laminated spring (6) that is attached to surface of the piston (2) that face the first compartment (4a) and covers the opening of the hole (5) at least partially. Spring (6) can be attached to the surface of the piston (2) by using a screw (3) or by some other way.

Hole (5) is lying along the surface of the piston (2) that faces the first compartment (4a) continuously or non-continuously through a curve around the center (M) and reaches the surface of the piston (2) that faces the second compartment (4b).

Laminated spring (6) is attached from aforesaid center (M) to the surface of the piston (2) that faces the first compartment (4a). Spring (6) harbors a part is placed at correct distance and has a shape that can cover the hole (5) completely from the center (M).

During the movement of the piston (2) which will compress the second compartment (4b), a resistance is formed against the increase of pressure in the second compartment (4b) against the movement of the piston (2). Meanwhile, the continuation of the movement is realized by fluid flow from the second compartment (4b) to the first compartment (4a) via hole (5). If the pressure difference exceeded a specific value, the spring (6) which covers the hole (5) partially leaves the surface of the piston (2) in a way that leads the complete opening of the whole because of the force exerted upon the spring (6) and fluid flow through the whole hole (5) is ensured.

With the realization of additional fluid flow when the pressure difference exceeds a specific value; effects like resistance profiles that depends of the exerted pressure upon the piston (2) according to the protection of the piston (2) and cylinder (1) from high pressure and requirement of area of utilization can be obtained.

Since the fluid flow rate from the second compartment (4b) to first compartment (4a) depends on the dimension of hole (5), the resistance that will be formed against the movement of the piston (2) is also depended on the dimensions of hole (5). Different effective apertures (5a) can be obtained for every valve by attachment/placement of standard spring (6) at different angular locations according to the center (M) on a piston (2) that is manufactured with a standard hole (5). in a way that will cover the specific amount of the effective range (5a). Thus every manufactured valve can put resistance against the movement of the piston (2) at different predefined levels.

IN the situation when the spring (6) covers the hole (5) completely, an effective range (5a) is not present. Then, in the situation when a force exerted to piston (2) is in a magnitude that can't cause the formation of a pressure that allows the opening of the hole (5) by spring (6), piston (2) only moves in a level of compressibility of the liquids.

Even at least the partial coverage of the hole (5) opening by the spring (6) above, it is also possible that the spring (6) does not cover the hole (5) at all. But in that situation, effective aperture (5a) will be as big as the hole (5) and the movement of the spring (6) won't have any effect on the piston (2).

In the situations when the hole (5) is lying along the curve continuously, the angular difference between the dimension of effective aperture (5a) and different angular positions of the spring (6) is related linearly. In the situation when the hole (5) is lying non-continuously, the dimension of effective aperture (5a) changes gradually.

The pressure difference value that the spring (6) allows the complete usage of the hole (5) rather than the effective aperture (5a) by opening, can be adjusted by changing of the spring constant and/or utilization of springs (6) with different pre-tension levels.

With the increasing stiffness of the spring (6), the required amount of pressure difference value also increases. For utilization of springs (6) with different pre-tension levels, spring (6) can be attached to the surface of the piston (2) in a position that spring (6) can be compressed by this surface while in loose position.

In one embodiment of present invention spring (6) is in a shape of a planar spiral. Spring (6) consist of central sheet (7) that forms the internal end and located at the center (M) of the spiral and the spiral arm (8) that contains a free end. Central sheet (7), is attached to the surface of the piston (2) faces to the first compartment (4a). Spiral arm (8) is touched to the aforesaid surface and covers the opening of the hole (5).

Piston (2) of present invention contains at least one valve containing laminated spring (6). In one embodiment of the present invention, at least two opposite oriented valves are present on the piston (2). In other words, piston (2) contains at least one valve that contains a spring (6) on surface of the piston (2) that faces to the first compartment (4a) and at least one valve that contains a spring (6) on surface of the piston (2) that faces the second compartment (4b), totally two valves. Thus, protection of cylinder (1) and piston (2) from the high pressure during the movement of the piston (2) in two directions and different resistance profiles depended to the exerted force upon the piston (2) according to the area of utilization can be obtained.

According to the invention that allows fluid flow in both direction, with the exceeding of a specified pressure difference during a movement of a piston (2) which compresses the second compartment (4b), spring (6) that remain in the first compartment opened the related hole (5) allow the fluid flow from the second compartment (4b) to the first compartment (4a) faster than the effective aperture (5a). During the movement of the piston (2) in the opposite direction, with the exceeding of a specified pressures a result of compressing of the first compartment (4a), the spring (6) that remain in the second compartment (4b) opened the related hole (5), allow a fluid flow in opposite direction, from the first compartment (4a) to the second compartment (4b) faster than the effective aperture (5a). During the movement of the piston (2) in different directions, with the exceeding of different pressure differences, the opening of the valves can be provided. For this, the valves should include springs (6) with different spring orientations.

In one embodiment of present invention that allows the fluid flow between compartments with different velocities, valve of the invention allows the fluid flow between a first compartment (4a) and a second compartment (4b) separated by a piston (2) which is placed in a cylinder (1) and can move in both directions on the axis of this cylinder (1) during the operation of the piston (2). Valve contains at least one hole (5) which is placed on the piston (2) and allows fluid flow between first compartment (4a) and second compartment (4b) and two laminate springs (6) which is attached to surfaced of the piston (2) that faces the either compartments and at least partially covers the both of the openings of the hole (5) that facing the both compartments. These springs are first spring (6a) which is attached to the surface of the piston (2) that faces the first compartment (4a) and related to the opening of the hole (5) that faces the first compartment (4a) and second spring (6b) which is attached to the surface of the piston (2) that faces the second compartment (4b) and related to the opening of the hole (5) that faces the second compartment (4b). Springs can be attached to the piston (2) surface by using a screw (3) or any other method.

Each of the first spring (6a) and second spring (6b) contains a part which has a shape that at least completely covers the hole (5) along a circle in which the center (M) is the attachment point and pass along the opening of the hole (5) on the related surface of the piston (2) and a portion which is adjacent to this portion and its radius is reduced in a way it don't cover the hole (5) at all by moved away in the azimuth direction.

During motion of the piston (2) which is in the direction of compression of the second compartment (4b), it consists of a resistance against the movement of the piston because of the high pressure. Meanwhile, the motion is maintained with the fluid flow from the second compartment (4b) into the first compartment (4a) through the hole (5). If the pressure difference exceeds a certain value, with the influence of the force acting on first spring (6a) which covers the opening of hole (5) facing the first compartment (4a), first spring (6a) is separated from the piston (2) while opening of the mentioned opening of the hole (5) and fluid flow is ensured through the whole part of the mentioned opening.

Motion of the piston (2), which is in the direction of compression of the first compartment (4a), takes place in a similar manner. If the pressure difference exceeds a certain value during this motion, with the influence of the force acting on second spring (6b) which covers the opening of hole (5) facing the second compartment (4b), second spring (6b) is separated from the piston (2) while opening the mentioned opening of the hole (5) and fluid flow is ensured through the whole part of the mentioned opening.

If the pressure difference exceeds a certain value, excess amount of fluid flow is provided; so that it is possible to obtain effects like protection of the cylinder (1) and the piston (2) from the high pressure, profiles adopting to the changing applied force according to the needs of usage area.

Because the fluid flow rate between compartments depends on the size of the hole (5), resistance against the movement of the piston (2) also depends on the size of the hole (5). During the valve production it is possible to obtain different effective aperture (5a) sizes by connecting/positioning of a piston (2) which have a standard hole (5) to a standard spring (6) that covers certain parts of the opening on both sides of the hole (5) with different angular positions. The valves that are manufactured in this way can resist against the motion of the piston (2) with different predefined levels. First springs (6a) and second springs (6b) can cover the mentioned hole (5) openings in different proportions respectively. In this case, effective aperture (5a) is equal to the area of the hole (5) opening which could not be covered by the springs.

There is no effective aperture (5a) when one of the springs cover the hole (5) completely. In this situation, when a force affects the piston (2) that is big enough to form a pressure which cause the spring (6a) to open the hole (5), the piston (2) will move in levels related with the compressibility of the fluid. In the case of when both first (6a) and second (6b) spring close the hole (5) completely, independent of the pressure difference between the first compartment (4a) and second compartment (4b), the piston (2) will move solely in a level related with the compressibility of the fluid.

It is possible that the springs never close the opening of the hole (5). Nevertheless, in this case effective tange (5a) will have same size with the hole (5) and there will be no influence of the springs on the motion of the piston (2).

Pressure difference value, in the case of when only one of the springs were opened and the other spring define the effective aperture (5a), is possibly adjusted by changing the spring constant and/or using other springs with different pre-stress levels. The pressure difference value will increase, when the stiffness of the spring is increased. To benefit from the different pre-stress levels, the spring may be connected to the related face of the piston (2) while it is in the free position, which can be compressed by this surface.

Preferably both connection points of the first (6a) and second (6b) springs are located on the intersection of a single parallel line to piston (2) surface and axis of cylinder (1). Thus, especially in the applications where springs are connected to the piston (2) through a screw (3), process of opening screw (3) holes on the piston (2) can be achieved in an easier and reliable way.

The hole (5) can reach to a opening or a curve on the other surface of piston (2) along a curve that goes continuous or discontinuous through the perimeter of a center (M) on the surface of piston (2). In this case, preferably, center (M) of the mentioned curve overlaps with the connection point of the spring which is connected to the related piston (2) surface.

In an embodiment of the present invention each spring is in the shape of a planar spiral. The spring is comprised of a centrally located central sheet (7), which forms inner end and, and a spiral arm (8) which posses a free end. The central sheet (7) is connected to surface of the piston (2). The spiral arm (8) is touching to the related surface and covers one of the hole (5) openings.

According to the present invention, the piston (2) possesses at least one valve that has two laminated springs (6) and these springs covers two openings of the hole (5) at least partially. During the motion of the piston (2) in the direction of compressing the second compartment (4b), the first spring (6a) in the first compartment (4a) opens the related hole (5) after a certain amount of pressure was overcomed and it allows faster fluid flow than the effective aperture (5a) from the second compartment (4b) to the first one (4a). On the other hand during the opposite motion of the piston (2) with the compression of first compartment (4a), the second spring (6b) inside the second compartment opens the related hole (5) after a certain amount of pressure was overcomed and it allows faster fluid flow than the effective aperture (5a) in the reverse direction namely from the first compartment (4a) to the second compartment (4b). During motions of the piston (2) on different directions, overcoming certain amounts of pressure allows opening of the springs. For these type of motions, it is sufficient that the valve has springs with different spring constants.

In an embodiment of the present invention, to enable the dynamic control of fluid flow between the compartments and die flow rate, the related valve is composed of a piston (2) that is located inside a cylinder (1) and it can move in both directions along the cylinder (1) axis and this piston (2) separates the first (4a) and second compartments (4b), so during movement of piston (2) fluid flow established. The valve contain at least one channel (9) that is located inside the piston (2) and provide fluid flow between first (4a) and second compartment (4b), a valve spring that can cover the opening of the channel (9) which faces towards the first compartment (4a), and an motor (10) that changes the position of the spring (6) so it can control the fluid flow and/or flow rate. The motor (10) can change the position of spring, in a way that will partially or completely cover the opening of the channel (9). In an application of the invention, the motor (10) can change the position of the spring (6) which never close the channel (9) opening.

The spring (6) is placed on the face of piston (2) which faces to the first compartment (4a) in a way that can rotate over a center (M). The spring (6) contain a part that go through the opening, which opens to the first compartment (4a) of channel (9) and along a circle which encircles the center (M), so that this closure part (11) and the gap (12) close the mentioned opening completely. The spring (6) could be placed in angular positions which can cover the channel (9) opening completely or partially. The option of changing the springs (6) angular position with the motor (10) let to adjust fluid flow and flow rate. The spring (6) may contain a gap (12) in a certain size that will never close the mentioned opening. In this case, the spring (6) can be resided in angular positions which will never close the channel (9) opening with respect to the center (M).

Motor (10) can be directed according to varying need with the help of a remote controller by a user person or automatically after a result of an input related to the usage format. Moreover, the motor (10) can be directed continuously by a software that is uploaded to controller unit according to fluid pressure or during motion of the piston (2) according to its position. Thus, it is possible to control the fluid flow and flow rate dynamically for different usage area and a number of different profiles.

In an embodiment of the present invention, the spring (6) contain a partially covering portion (13) which close the related channel opening (9) partially as well as the covering portion (11) and the gap (12). With a spring (6) which does not have a partially covering portion (13), it is possible close the channel (9) opening partially, however in this application of the invention during the process of partially closing the channel opening the closure percentage can be adjusted sensitively.

In a preferable embodiment of the present invention, the motor (10) is embedded inside the piston (2) shaft (14) with purposes of protecting the motor (10) from fluid and connecting the motor (10) to a remote controller or power supply easily. In this version of invention, the channel (9) opening, which opens to the first compartment (4a), is not connected directly to the first compartment (4a) whereas connected with an intermediate compartment (15) and with a hole (5) that resides between intermediate compartment (15) and the first compartment (4a). The motor (10), which is embedded inside the shaft (14), is separated from the intermediate compartment (15) with a gasket (16). A transmission shaft (17), which pass through inside the gasket (16) and connects the motor (10) and the spring (6), enables the angular position shift of the spring inside the intermediate compartment by the motor (10).

During the motion of the piston (2) which compress in the direction of first compartment (4a), there is fluid flow from the first compartment (4a) to the second compartment (4b). Meanwhile the fluid, which passes through the hole (5) on the shaft (14) to the intermediate compartment (15), form pressure on the spring (6) and cause the spring (6) to sit on the channel (9) opening facing the first compartment (4a). Thus, flow rate and accordingly the resistance, which is formed against the piston (2) motion, is determined by the angular position of spring (6) that is adjusted with motor (10) activity. Moreover, when the pressure inside the second compartment (4b) increases and pressure difference between first compartment (4a) and second compartments (4b) exceeds a certain value; namely when the force that is acted on the spring (6) by the pressure from the second compartment (4b) is higher than the total force that is acted on the spring (6) from the first compartment (4a) and the force that the spring (6) has formed, the spring seperates from the piston (2) surface in a way that opens the channel (9) mouth completely and fluid flow is established fully on the channel (9) with the effect of the force that is acted on the spring (6) which partially covers channel (9) mouth. Thus the valve that is the subject of the invention work as a pulling valve as well as a resistance valve.

If the pressure difference exceeds a certain value, excess amount of fluid flow is provided; so that it is possible to obtain effects like protection of the cylinder (1) and the piston (2) from the high pressure, profiles adopting to the changing applied force according to the needs of usage area.

The invention was explained for a leaflet of spring (6), however a valve can be obtained via a plaque which have a covering portion (11) and a gap (12) that passes through the first compartment (4a) facing channel (9) opening and covers at least the related opening completely along a circle around the center (M), so these parts can be used to control fluid flow and flow rate with the help of the motor (10). But a valve like this, which have a plaque instead of a leaflet spring (6), will not permit acceleration of fluid flow during sudden increases in fluid pressure. The gap (12) may be in a size of that will never cover the related opening.

In another embodiment of the present invention, again to enable the dynamic control of fluid flow between the compartments and the flow rate, the related valve set is composed of a piston (2) that is located inside a cylinder (1) and it can move in both directions along the cylinder (1) axis and this piston (2) seperates the first (4a) and second (4b) compartments, so during movement of piston (2) fluid flow established. The valve set contains two channel (9) that is located inside the piston (2) and provide fluid flow between first (4a) and second (4b) compartment, two valve springs (6) that can cover the opening of the channels (9) which faces towards the first compartment (4a) and second (4b) compartment, and at least an motor (10) that changes the position of the spring (6) so it can control the fluid flow and/or flow rate. The motor (10) can change the position of spring (6), in a way that will partially or completely cover the opening of the channel (9). In an application of the invention, the motor (10) can change the position of the spring (6) which never close the channel (9) opening.

The springs (6) can be controlled by a single motor (10) through a transfer organ, and each spring (6) preferably is controlled by a separate motor (10). By the virtue of the usage of the two motors (10), the springs (6) can be controlled independently from each other.

Each spring (6) is placed on the related face of piston (2) in a way that can rotate over a center (M). Each spring (6) contain a part that go through the opening of the related channel (9) along a circle which encircles the center (M), so that this covering portion (11) and a gap (12) close the mentioned opening completely. The springs (6) could be placed in angular positions which can cover the channel (9) opening completely or partially. The option of changing the springs (6) angular position with the motor (10) let to adjust fluid flow and flow rate. The springs (6) may contain a gap (12) in a certain size that will never close the mentioned opening. In this case, the spring (6) can be resided in angular positions which will never close the channel (9) opening with respect to the center (M).

Motors (10) can be directed according to varying need with the help of a remote controller by a user person or automatically after a result of an input related to the usage format. Moreover, the motors (10) can be directed continuously by a software that is uploaded to controller unit according to fluid pressure or during motion of the piston (2) according to its position. Thus, it is possible to control the fluid flow and flow rate dynamically for different usage area and a number of different profiles.

In an embodiment of the present invention, each spring (6) contain a partially covering portion (13) which close the related channel (9) opening partially as well as the covering portion (11) and the gap (12). With a spring (6) which does not have a partially covering portion (13), it is possible close the channel (9) opening partially, however in this application of the invention during the process of partially closing the channel opening the closure percentage can be adjusted sensitively.

In an embodiment of the present invention, there is only one channel (9) instead of two channels (9). In this case, one of the springs (6) resides in a position of covering the channels (9) one opening, and the other spring (6) covers the other opening.

In a preferable embodiment of the invention, the motors (10) are embedded inside the piston (2) shaft (14) with purposes of protecting the motors (10) from fluid and connecting the motors (10) to a remote controller or power supply easily. In this version of invention, the channel (9) openings, which are covered by the springs, is not connected directly to the related compartment whereas connected with an intermediate compartment (15) and with a hole (5) that resides between each intermediate compartment (15) and the first compartment (4a) or second compartment (4b). The motors (10), which are embedded inside the shafts (14), is separated from the intermediate compartments (15) with a gasket (16). Transmission shafts (17), which pass through inside the gasket (16) and connect the related motor (10) and the related spring (6), enable the angular position shift of the springs inside the intermediate compartment by the motors (10).

During the motion of the piston (2) which compress in the direction of first compartment (4a) or second compartment (4b) for example there is fluid flow from the first compartment (4a) to the second compartment (4b) while the movement of the piston (2) compressing the first compartment (4a). Meanwhile the pressure created by fluid that passes from hole (5) on the shaft (14) to intermediate compartment (15) on spring (6) which covers the channel (9) opening provides the spring (6) to fit into the related channel (9) opening. Thus fluid flow thereby resistance against to the movement of piston to be identified with springs (6) of which angular position adjusted via motors (10) and getting effects like limitation of the direction of movement, limitation of the speed of movement. By means of continuous controlling both of the springs' (6) positions provides an effective control over the piston's (2) movement. Moreover, when the pressure inside the second compartment (4b) increases and pressure difference between first (4a) and second (4b) compartments exceeds a certain value, namely when the force that is acted on the spring (6) by the pressure from the second compartment (4b) is higher than the total force that is acted on the spring (6) from the first compartment (4a) and the force that the spring (6) has formed, the spring seperates from the piston (2) surface in a way that opens the channel (9) opening completely and fluid flow is established fully on the channel (9) with the effect of the force that is acted on the spring (6) which partially covers channel (9) opening. Thus the valve that is the subject of the invention work as a pulling valve as well as a resistance valve.

In an another embodiment of the present invention, the motor (10) again is embedded inside piston (2) shaft (14) but the channels (9) are not lie directly between the first compartment (4a) and second compartment (4b). They lie between first compartment (4a) and a common intermediate compartment (15) and also between second compartment (4b) and common intermediate compartment (15). The springs (6) resides in the common intermediate compartment (18) in a way that they can cover the faces of channels (9), which are directed toward common intermediate compartment. FIG. 31 shows a drawing of this version of the invention. It shows tone each motors (10) that are placed in two sides of the piston (2) and in the middle of those the common intermediate compartment (18) inside the piston (2). In spite of that, in this version of the invention, it is possible to form the common intermediate compartment (18) inside the shaft (14) in different positions by extending at least one of the channels (9) inside the shaft (14).

When the common intermediate compartment (18) is used, because of high pressure in the first compartment (4a) or second (4b) compartment the springs (6) will be pushed by the fluid inside the common intermediate compartment (18) toward the opening of the channels (9); so that in the case unwanted high pressure values the excess fluid flow operation cannot be performed. Accordingly in a version of the invention which contain common intermediate compartment (18), there exist a high pressure division connected to the common intermediate compartment (18). High pressure division, for instance, may contain a high pressure piston (2) and another second fluid that interact with the regular fluid via this high pressure piston (2).

The invention was explained for leaflet of springs (6), however a valve set can be obtained via a plaque which have a covering portion (11) and a gap (12) that passes through the first compartment (4a) or second compartment (4b) facing channel (9) opening and covers at least the related opening completely along a circle around the center (M), so these parts can be used to control fluid flow and flow rate with the help of the motor (10). But a valve set like this, which have a plaque instead of a leaflet spring (6), will not permit acceleration of fluid flow during sudden increases in fluid pressure. The gap (12) may be in a size of that will never cover the related opening.

The control method, which is subject of the invention, provides dynamical management of profiles determined according to the explained valve set which is used for different usage areas of fluid flow and flow rate. This method basically includes these steps;

(101) Receiving the data related to pistons (2) motion phase

(102) Determining the angular positions of each spring (6) that corresponds to this motion phase.

(103) Powering the motors (10) up to adjusting the springs (6) at the determined angular positions.

These steps are repeated during the motion. Repeating frequency can be determined according to the usage area of the valve set.

Pistons (2) motion phases can be defined by using pistons (2) position and motion direction. The data related to the motion phase can be received with the systems like a sensor which measures the pistons (2) position as well as comparing this with the previous measure result, a sensor on a machine that moves the piston (2) or a control unit which directs this machine, or pressure sensors inside the first compartment (4a) and second compartment (4b) that compare the expected pressure values with the measured ones.

The angular positions of each spring (6) which corresponds to the movement phases can be kept as a registered value in a table or as mathematical relations which make the calculation during the movement is possible. In case the angular positions are kept as a table, the highest control sensitivity is dependent upon the number of entries in the table. In case the angular positions are kept as mathematical relations, the highest control sensitivity is dependent upon the calculation speed.

The dynamical control of the fluid flow and the fluid flow rate can be possible by a regular update of the angular positions of the springs (6). The resistance against the piston (2) movement may change depending on the fluid flow and the flow rate. Thus the piston (2) can function in a way that encounter a resistance which depends on pistons (2) motion phase.

In an embodiment of the present invention, to prevent the damage may be done at the piston (2) motions end points, it is possible to increase the resistance when the piston (2) come closer to these points.

The resistance against the piston (2) during its motion can change with the motion phase as well with the other factors. For instance; the resistance can be adjusted once again according to the other factors which shows variety with pistons (2) motion velocity or usage area. Because of this, between the steps 102 and 103; the step of adapting the angular positions, which were defined in step 102, to at least for one factor (104). Adapting process can be achieved with a mathematical relation that includes a separate component for each factor which are related to the angular positions that defined in step 102. The components related to the factors may be constant as well as expressions that may change depending on the motion phase.

In another embodiment of the present invention the valve set stands over a first body (19), which is placed upon the end of a first prosthesis part that at least partially replaces a first bone, and a second body (20), which is placed upon the end of a second prosthesis part that at least partially replaces a second bone, that are connected to each other in a way to determine at least one rotational axis, a prosthetic joint that includes a second body (20) which is located at one end of the second prosthesis part; a cylinder (1) and a piston (2) which divides the inner volume of the piston (2) into two; and two compartment (4) that stay between the cylinder (1) and the piston (2) and also have a fluid that form a resistance against the angular motion of first prosthesis and second prosthesis parts relative to each other.

Cylinder (1) and piston (2) while first and second prosthesis part moving towards each other, cylinder (1) and piston (2) move linearly with respect to each other and changes compartments (4) volumes. So the fluid flow in the compartment (4) creates a resistance between cylinder (1) and piston (2) which leads to a resistance between first prosthesis part and second prosthesis part moves.

The valve set that is subject of the invention includes a intermediate compartment (15) which is embedded inside the piston (2), a channel (9) which connects each compartment (4) to intermediate compartment (15), holes (5) between the channels (9) and the intermediate compartment (15), plaque leaflet springs (6) which can cover the holes (5), and at least one actuator (21) which controls the fluid flow and the fluid rate by changing the angular positions of the springs (6).

The springs (6) can be controlled by a single actuator (21) through a transfer organ, and each spring (6) preferably is controlled by a separate actuator (21). By the virtue of the usage of the two actuators (21), the springs (6) can be controlled independently from each other.

Each hole (5) extends along a radial arc on the surface of intermediate compartment (15). Each spring (6), along a circle around the aforementioned center (M) which passes from the respective hole (5), includes a closer section which covers an area at least in size which can fully cover the aforementioned end and a gap (12) at least in size which does not cover the aforementioned opening at all. The springs (6) can rotate around the aforementioned center (M). By this way, the springs (6) may be at different angular positions which cover the aforementioned hole (5) partially or completely, or do not cover at all. By means of the change at these angular positions of the springs (6) through the actuators (21), the actuator fluid and the speed of the transition can be adjusted.

Actuators (21) can be driving shafts which are driven through the various transfer organs as a result of the manual movement of the user or of the angular movement of the first prosthetic part and the second prosthesis relative to each other, however the actuators (21) are preferably motors. The motors can be controlled according to the variable needs by a user via a control unit or automatically as a result of an entry as regards to the usage format.

The motors can also be managed in a continuous way during the movement of the piston (2) in accordance with the fluid pressure or piston (2) position by a program which is installed in the control unit. Thus, the fluid flow and the transition speed can be dynamically controlled according to the more than one profiles which are determined for the different usage areas. For instance, it is possible to make an upper leg prosthesis work in accordance with the different profiles of pacing during walking and running.

In a preferred embodiment of the present invention, the actuators (21) are positioned inside the cylindrical actuator beds (23) parallel to the shaft (14) which is opened in a first end (22) of the piston (2) shaft (14). The connections to the units such as control unit and power supply can be done through the parts of the actuators (21) which extend from the first end (22) of the shaft (14) to the outside. The springs (6) are directed by the sections of the actuators (21) which stay inside the shaft (14). A groove (25), which is opened on a second end (24) of the shaft (14) and which intersects with the actuator beds (23), interconnects the actuator beds (23). The intermediate compartment (15) is consist of the bottom of the actuator beds (23) along with the volume and the groove (25) which remains between the actuators (21). Channels (9) are comprised of a cylindrical first part (26), which is opened at the first end (22) of the shaft (14) and which is parallel to the shaft (14), a second part (27), which connects each first parts (26) to one of the compartments (4), and a cylindrical third part (28), which is opened at the second end (24) of the shaft (14) parallel to the shaft (14) and in which each of them intersects with one of the first parts (26). The holes (5) provide a connection between the actuator beds (23) and third parts (28). In this application of the invention, since the openings of the actuator beds (23), the channels (9) except the second parts (27) and the intermediate compartment (15) are carried out at the first end (22) and second end (24) of the shaft (14), a piston (2) which includes the valve set, which is the invention subject, can be easily produced.

For the sake of preventing fluid leakage from the first parts (26), the third parts (28) and the groove (25), there exists covers (29) which are placed upon the first end (22) and the second end (24) of the shaft (14). The sealing gaskets (16) between the actuators (21) and the intermediate compartments (15) are present to prevent the actuators (21) from the leakage. A transfer shaft, which is connected to the respective actuator (21) at the one end and to the respective spring (6) at the other end via passing through each gasket (16), ensures that the angular positions of the springs (6) which stay inside the intermediate (15) can be changed by the actuators (21).

Piston (2) compresses one of the compartments (4) during its movement in one direction. A resistance against the movement of the piston (2) occurs because of the increased pressure at the compressed compartment (4). At the same time, a fluid flow which is obtained by the holes (5) ensures that the resistance is kept at the desired level. Since the fluid flow is determined by the angular positions of the adjusted springs (6) through the actuators (21), effects such as the limitation of the movement in one particular direction and the limitation of movement speed can be obtained. By the means of control over the angular positions of both springs (6), the movement of the piston (2) can be directed efficiently.

Prosthetic joint which is the subject of the invention contains; a piston (2) which is connected to the first body (19) at the joint of the first body (19) and the second body (20), in other words, at the rotational axis, from the first end (22) of the shaft (14) and the second end (24) of the shaft (14); a cylinder (1) which moves linearly with respect to the piston (2) along the rotational axis by interacting with the first body (19) during the movement of the first prosthesis section and second prosthesis section in relation to each other; and a valve set, according to the invention, which ensures a resistance by the liquid at the compartments (4) between the cylinder (1) and the piston (2) during the movement of the cylinder (1) in relation to the piston (2). By the help of this resistance, the relative movement of the first prosthesis part and the second prosthesis part can occur in accordance with the natural movement of the limb which is replaced by the prosthesis.

The interaction between the cylinder (1) and the second body (20) is ensured by spiral shaped teeth (30) which are placed on the curved surface of the cylinder (1) and by spiral counter teeth (31) which are located at the inner surface of the extension of the first body (19) which covers the cylinder (1). During the movement of the first prosthesis part and the second prosthesis part in relation to each other, the cylinder (1) and the first body (19) are in angular motion as well in relation to each other and the cylinder (1) is forced by the counter teeth (31) through teeth (30) to be in linear motion too along the rotational axis.

The cylinder (1) is consist of three parts in total in which one of them comprise the curved surface and two of them include the flat surfaces. By this way, the montage of the cylinder (1) can be easily carried out.

In one of the embodiment of this invention, cavities on the teeth (30) which are parallel to the rotational axis and the counter teeth (32) at the inner surface of an extension of the second body (20) which covers the cylinder (1) are present. The cavities and linear counter teeth (32) ensures that the cylinder (1) is supported during its linear movement along the rotational axis.

The joint prosthesis, which is the subject of the invention, can be used as a replacement for the knee joint in the leg prosthesis or as a replacement for the elbow joint in the arm prosthesis. Especially during the various activities including the applications in which the prosthetic joint is used for the replacement of the knee joint such as for walking, running, climbing up stairs, climbing down stairs, sitting, the efficient orientation of the movement of the lower leg in accordance with the upper leg is provided, and the gap and the speed of the releasing of the lower leg in accordance with the phase of the natural movement can be adjusted sensitively. Thus, it is possible for the user to move in a natural way.

The subject joint prosthesis of the invention can also include more than one rotational axis. For instance, in a knee joint in which there is four-rod mechanism, a cylinder (1) and a piston (2) set, which are place along one or more rotational axises, can be present.

Prosthetic joint which is the invention subject can be dynamically controlled in accordance with the profiles which correspond to the pre-determined movement types for the various activities by the steps of,

(201) the collection of the data as regards to the phase of the movement in which the for is applying,

(202) the determination of the angular positions of each spring (6) which corresponds to this movement phase

(203) the running of the motors to bring the springs (6) into the determined angular positions.

The movement phases can be determined by the measurement of the angle between the first prosthesis part and the second prosthesis part, the angular position of the cylinder (1) or the linear position of the cylinder (1).

The angular positions of each spring (6) which corresponds to the movement phases can be kept as a registered value in a table or as mathematical relations which make the calculation possible during the movement. If the angular positions are kept as a table, the highest control sensitivity is dependent upon the number of entries in the table. If the angular positions are kept as mathematical relations, the highest control sensitivity is dependent upon the calculation speed.

The dynamical control of the fluid flow and the fluid flow rate can be possible by a regular update of the angular positions of the springs (6).

During a particular activity, in case the user faces an obstacle which prevents one of his/her movement, the adaptation of the movement profile of the prosthetic joint might be necessary. For instance, the user may pass through a ground which has variable slopes. If this is the case, in between 202^{nd} and 203^{rd}, the step (204), which defines the adaptation of the angular positions which are determined at the 202^{nd} step according to the at least one factor, can be applied.

We claim:

1. A valve, that is placed inside a cylinder, ensures a fluid flow between a first compartment and a second compartment which are divided by a piston that moves in two directions along a cylinder axis,
comprising,
at least one hole which extends along an are around a center at a surface of the piston which faces the first compartment;
a laminated spring which is mounted to the piston which at least partially covers an opening of the hole;
wherein:
the hole extends along the arc around the center at the surface of the piston which faces the first compartment;
the laminated spring which is mounted to the piston at the center includes a part having an area and form to fully cover the hole by standing in an appropriate distance from the center,
and wherein the laminated spring connected/located in a plurality of different angular positions according to the center in a way to cover a specified part of the hole.

2. The valve according to claim 1, wherein the hole extends continuously along the arc around the center.

3. The valve in according to claim 1, wherein the hole extends discretely along the arc around the center.

4. (canceled)

5. The valve according to claim 1, wherein each of the laminated springs is a junction in which a respective central laminated spring is fixed, and contains a part covering an area in size at least to fully close the hole and another adjacent part which covers an area that decreases until there is no cover for the hole.

6. The valve according to claim 5, wherein both junctions of the laminated springs are placed upon an intersection of the piston and a single line parallel to the cylinder axis.

7. The valve according to claim 1, further comprising
at least one channel which exists in the piston that ensures the fluid flow between the first compartment and the second compartment,
a leaf spring which closes the opening of the channel which is opened to the first compartment, wherein
the first compartment (4a) and the second compartment (4b) are separated from each other by the piston, wherein a motor controls a fluid flow and/or a fluid flow rate by changing a position of the laminated spring.

8. The valve according to claim 7, wherein the laminated spring contains a closing part which passes through the opening of the channel which is opened to the first compartment, which covers an area in size to fully close the opening along the circle around the center covering portion, and a gap.

9. (canceled)

10. (canceled)

11. (canceled)

12. The valve according to claim 7, wherein the motor is embedded inside a shaft of the piston.

13. (canceled)

14. The valve according to claim 7, further comprising
two channels which exist in the piston which provides the passage of the fluid between the first compartment and the second compartment,
laminated springs which closes mouths of the one of the two channel that faces the first compartment and the other channel that faces the second compartment,
which includes a covering portion which covers an area in size to fully close the mouth along the circle around the center and a gap, the covering portion and the gap along the circle around the center which passes the respective mouth of the channel, wherein at least the motor which controls the fluid flow and/or the fluid flow rate by changing the position of the spring, the first compartment and the second compartment are separated by the piston.

15. The valve according to claim 14, wherein a separate motor controls each of the laminated springs.

16. (canceled)

17. (canceled)

18. (canceled)

19. The valve according to claim 14, wherein a high pressure section is connected to a common intermediate compartment that contains a high pressure piston and a compressible second fluid which interacts with the fluid through the high pressure piston.

20. A control method which ensures the control of the valve of claim 14, comprising followings steps:
collecting data as regards to a phase of a movement in which the piston is applied;
determining angular positions of each laminated spring which corresponds to movement phase;
running the motors to bring the laminated springs into determined angular positions,
repeating the above listed steps during the piston movement.

21. The control method according to claim 20, wherein an adaptation step, which is achieved before running the motors, of the determined angular positions in accordance with at least one factor.

22. (canceled)

23. (canceled)

24. (canceled)

25. The control method according to claim 20, wherein increasing resistance that the piston faces when the piston approaches extremes of the movement.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A piston comprising at least two valves in claim 1, and at least one of the two valves includes a first spring which is placed on the surface of the piston facing the first compartment and at least one of the two valves includes a second spring facing the second compartment.
